(19) United States
(12) Patent Application Publication
(10) Pub. No.: US 2021/0081472 A1
(43) Pub. Date: Mar. 18, 2021

(54) GENERATING ONLINE SHARE SLIPS WITH CLICKABLE WEB LINKS FOR ELECTRONIC SHARE TRANSFERS FOR PREFERENTIALLY MATCHED MEMBERS WITH AFFINITY-BASED ALLOCATION

(71) Applicant: SAMARITAN MINISTRIES INTERNATIONAL, Peoria, IL (US)

(72) Inventors: Seth Ben-Ezra, Peoria, IL (US); James Lansberry, Peoria, IL (US)

(73) Assignee: SAMARITAN MINISTRIES INTERNATIONAL, Peoria, IL (US)

(21) Appl. No.: 17/247,120

(22) Filed: Nov. 30, 2020

Related U.S. Application Data
(63) Continuation of application No. PCT/US20/22635, filed on Mar. 13, 2020.
(60) Provisional application No. 62/819,201, filed on Mar. 15, 2019.

(51) Int. Cl. G06F 16/958; G06F 16/951; G06F 16/957; H04L 29/08; G16H 10/60

(52) U.S. Cl. CPC G06F 16/958; G06F 16/951; G16H 10/60; H04L 67/1044; G06F 16/957

(57) ABSTRACT

A process and system for allocating shares to needs within a peer-to-peer needs sharing organization. Shares are matched through an iterative process when an affinity-basis of the sending member matches or is compatible with the affinity-basis of a receiving member. The system preferentially assigns available shares to a selected need request if the first sending share has a first affinity basis compatible with a selected receiving affinity-basis of the selected need request. The affinity basis may include physical proximity, medical history, or payment capabilities, among others disclosed.

### CROSS REFERENCES

This application claims the benefit of U.S. Provisional Patent Application No. 62819201, filed 15 Mar. 2019, which is incorporated by reference herein in its entirety.

### FIELD OF THE INVENTION

The present disclosure relates to generating online share slips with clickable web links for electronic share transfers for preferentially matched members with e-service capabilities, enabling an increased number of members in a peer-to-peer health care needs sharing organization to engage in electronic share transfers.

### BACKGROUND

Health care sharing is an alternative to health care insurance products. Health care sharing is a type of a mutual benefit society where members commit to sharing in a portion of the medical needs of the other members. An organization can take an active role in receiving and disbursing the funds or a more passive role by providing share information to the members to engage in a peer-to-peer direct sharing model.

In a health care sharing model, generally, a member pays a periodic share, commonly monthly. When a member has a qualifying health care expense, the member may submit that expense as a need. The health care sharing organization allocates one or more other members to meet that need. The need can be met by the other members directly, in a peer-to-peer direct sharing model, or indirectly where the members send their share money to the health care sharing organization to collectively pool and distribute to the member in need.

### SUMMARY

E-Sharing provides members the opportunity to send and receive shares through a payment platform they may already use in addition to sharing through postal mail. Sharing electronically is possible when both the sending and receiving member use the same payment platform.

I discovered a process to preferentially connect members who had a matched or compatible share transfer method during the periodic allocation of shares. Some members are more technologically savvy, using computers, cell phones, tables, and other network connected electronic devices to communicate and manage money. Other members are less technologically savvy or merely have a preference for paper mail and checks. By preferentially displaying a graphical online share slip with a clickable web link to members set up to receive the shares via a digital, network-based solution to other members with similar capabilities, the overall membership has a better, more efficient sharing experience.

I also recognized that the time for a member to receive the shares may be decreased when members who are able to pay their shares electronically are matched with member desiring to receive shares electronically. Paper mail is slower than electronic mail, and sending checks through the mail is slower than sending funds using an online transfer service. In order for a member with a need to receive a faster online transfer, the member with the need is setup to receive the online transfer and the member sending the share is setup to send the online transfer. If the receiving member is not setup to receive the online transfer, then a member willing to send a share through online transfer is unable to take advantage of the faster online transfer. Similarly, if a member with a need is setup to receive an online transfer but the share sending member is not setup for online transfers, then the share must be transferred by postal mail.

Members may add and edit their payment links through a member portal having a graphical user interface. The member selects their preferred e-service payment method and enters their individualized payment links for one or more e-services. When the member has a need, the sharing members receive a share slip through email or the member portal. The share slip has a clickable web link to send the Share electronically instead of through postal mail.

I discovered that preferentially matching groups having the largest amount of total need dollars ensures that a greater number of members receive a share slip with a clickable web link to initiate an electronic payment of their share. The needs are sorted by total group dollar. The sharing preference group having the highest total dollar amount is selected. The largest needs within the need group having the highest total dollar amount is selected for allocation. The largest share, or the most closely matching share size, may be matched to the selected need. In this way, a larger number of sharing members receive an online share slip to send their share through an e-service.

I also recognized that maximizing the number of members sharing through electronic share transfer services minimizes the overall time between a member's need submission and receipt of all of their shares. The time to send a paper check through the mail is inherently time consuming: manually transposing the share amount from the share slip to the check, manually transposing the name and address of member to the check and envelope, affixing proper postage to the envelope, depositing the share in a mailbox, waiting for the postal service to deliver the share to the receiving member, and waiting for the member to retrieve the share from member's mailbox. The receiving member must then open the mail, manually complete a shares received checklist, and deposit the check with the member's bank. This can be especially burdensome when members have large needs, necessitating tens or hundreds of shares. Electronic share transfer systems can be almost instantaneous. As soon as the member clicks the appropriate web link the receiving user may receive the share.

Another advantage of the current disclosure is the generation of a web link that sends the share amount parameter to the online transfer service. When members send shares direct in the peer-to-peer model, the members commonly handwrite a personal check or manually enter the share dollar amount into an online bill payment or other check-printing interface. I discovered that displaying a web link in an online share slip allows the exact share dollar value to be automatically transferred to the online transfer service. By sending the share amount parameter through a clickable web link, the frequency of user error may be decreased compared with the member transposing numbers from a paper share slip on to a check.

Another advantage of the current disclosure is that the sending member does not have to manually transpose the receiving member's name and address correctly. When members send shares direct in the peer-to-peer model, the members commonly handwrite the receiving member's name and address on an envelope. I discovered that displaying a web link to a member's electronic sharing system profile enables the receiving member to control the receipt address through the electronic sharing system's profile. The receiving member can configure their electronic sharing system profile to deliver notices to their preferred email address and deposit electronically transferred funds to their preferred bank account. The receiving member can manage all of their received shares remotely. This enables a hospitalized member to manage the member's shares received from a hospital bed. Similarly, in situations where there is a natural disaster, the member may have medical needs arising from the natural disaster. The member may have to evacuate or be set up in a transient living situation, which can be difficult for the member to send checks or receive paper mail at a physical address. Maximizing the sending of shares through online share slips and electronic share transfers enables members to continue to participate in a health care sharing organization despite a transient living situation.

While the present disclosure is focused upon health care sharing, the same principles may apply to the sharing of other household expenses, income protection such as from disability, legal liability protection, and property protection.

It is understood that other embodiments will become readily apparent to those skilled in the art from the following detailed description, wherein various embodiments are shown and described by way of illustration only. As will be realized, the concepts are capable of other and different embodiments and their several details are capable of modification in various other respects, all without departing from the spirit and scope of what is claimed as the invention. Accordingly, the drawings and detailed description are to be regarded as illustrative in nature and not as restrictive.

### BRIEF DESCRIPTION OF DRAWINGS

Aspects are illustrated by way of example, and not by way of limitation, in the accompanying drawings, wherein:

FIG. 1 depicts a schematic illustration of peer-to-peer need sharing.

FIG. 2 depicts various matches between members who are or are not setup for electronic share transfers.

FIG. 3 depicts a schematic illustration of storing a member's share preference service priority.

FIG. 4 depicts a flow chart for the allocation engine preferentially matching members setup for electronic share transfers with receiving members setup to receive electronic share transfers.

FIG. 5 depicts a graphical user interface for an online share slip with clickable web links enabling the sending member to send a share through an electronic transfer service.

FIG. 6 depicts an additional information display and confirmation for the online share slip of FIG. 5.

FIG. 7 depicts an additional information display and confirmation for the online share slip of FIG. 5.

FIG. 8 depicts a schematic illustration of ranking multiple e-service providers according to a member preference rank.

FIG. 9 depicts a graphical user interface enabling a member to select a sharing preference between multiple e-services.

FIG. 10 depicts a flow chart for an allocation engine preferentially matching e-shares with e-needs and then matching regular shares with regular needs.

FIG. 11 depicts a flow chart for an allocation engine to allocate shares within a shares-analysis queue to selected needs from a needs-analysis queue.

FIG. 12 depicts a flow chart for an allocation engine to perform a matching set.

FIG. 13 depicts a flow chart for an allocation engine to preferentially assign shares from a share preference group matching the need before assessing other compatible share preference groups.

FIG. 14 depicts a flow chart for the allocation engine preferentially matching members setup for electronic share transfers with receiving members setup to receive electronic share transfers.

FIG. 15 depicts a flow chart for an allocation engine to determine the compatibility of the online share slip category as a compatible share preference group based on a selected need preference group.

FIG. 16 depicts a sample data set of qualifying member needs for a period.

FIG. 17 depicts a sample data set of sharing category associated with memberships.

FIG. 18 depicts a sample data set of need category associated with memberships.

FIG. 19 depicts a sample data set of an allocation having matched various shares from memberships to a need.

FIG. 20 depicts a flow chart for a process is disclosed for generating a share slip for each member of a peer-to-peer needs sharing community.

FIG. 21 depicts a flow chart for a process for allocation of shares to needs within a peer-to-peer needs sharing organization is disclosed as process.

### DETAILED DESCRIPTION

Allocation is the process of matching membership shares to member needs. The goal is to assign each share with a need. The allocation process is run periodically upon all of the needs submitted for the previous period. The period is commonly a monthly period, but could also be annually, quarterly, weekly, daily, or hourly depending on the number of members, regularity of needs, size of needs, and the members' need for prompt reimbursement. The number and amount of shares available to meet the needs may be affected by the number of members, the dollar amount of each member's share, deferment of a portion of the shares to the organization for administrative cost, and member credits. The allocation engine is a computer-based algorithm used to balance the shares and needs and to match each share with a need.

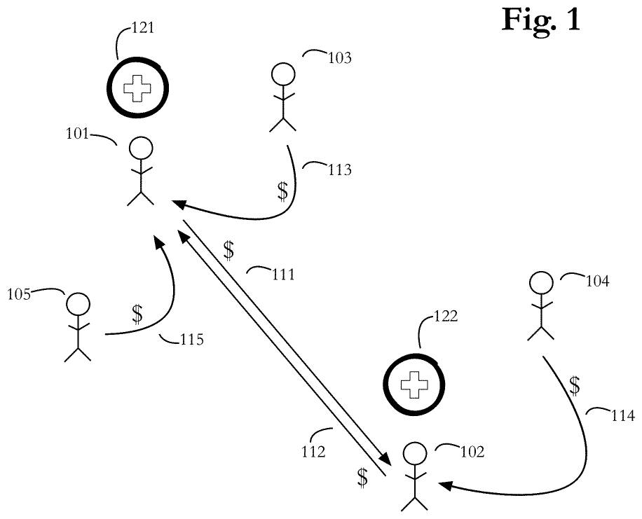

A hypothetical health care sharing organization is shown schematically in FIG. 1. The health care sharing organization comprises member 101, member 102, member 103, member 104, and member 105. Each member sends a share each period. Member 101 had medical need 121 and member 102 had medical need 122. Member 101 and Member 102 submit their need to the health care sharing organization. The health care sharing organization determines if the need qualifies for sharing under the organization's guidelines. The shares are allocated as follows: Member 101 sends share 111 to Member 102; Member 102 sends share 112 to Member 101; Member 103 sends share 113 to Member 101; Member 104 sends share 114 to Member 102; and Member 105 sends share 115 to Member 101. Each member sends a share to another member who had a need. This allows Member 101 and Member 102 to pay their medical expenses with the funds they receive from their fellow members. Each member bears a portion of the medical needs 121, 122.

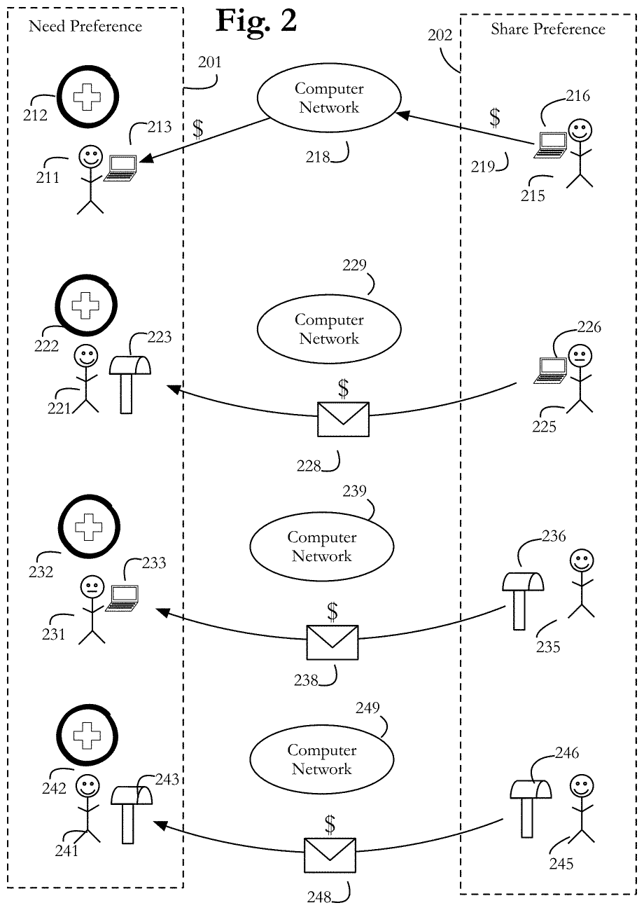

As shown in FIG. 2, a member can send or receive a share to another member in a variety of ways. Members having a need are shown in box 201. Members assigned to send a share are shown in box 202. Member 211 has medical need 212. Member 211 is setup to receive shares via an online transfer service, as indicated by icon 213. Online transfer services include Peer-to-peer payment applications and web portals like PayPal®, Square® Cash App™, PayPal® Venmo™, Google® Pay™, Apple® Pay Cash™. Banks may offer online transfer services using web portals or apps such as Zelle®. Member 215 is also set up for online transfer service, as indicated by icon 216. Member 215 share is allocated by the allocation engine to Member 211's medical need 212. Member 215 receives an online share slip enabling member to send the share electronically. Member 215 sends the share 219 through online transfer service via a computer network connection 218 between member 211 and member 215. Member 211 receives the share 219 through the online transfer service via the computer network connection 218.

When a member with a need is not setup to receive online share transfers, then a member sharing who is setup for online share transfers may be inconvenienced. Member 221 has medical need 222. Member 221 is not setup to receive shares via an online transfer service. Rather, Member 221 can only receive shares by paper mail, as indicated by icon 223. Member 225 is set up for online transfer service, as indicated by icon 226. Member's 225 share is allocated by the allocation engine to Member's 221 medical need 222. Member 225 may receive an online share slip, but it will not have a clickable web link to an e-service. Member 225 sends the share 228 by writing a check, manually transposing the share amount from the share slip to the check, transposing the name and address of member 221 to the check and envelope, affixing proper postage to the envelope, and mailing the check. After several days, Member 221 receives the share 228 through the paper mail service. Even though member 225 is setup for online transfer service through computer network connection 229, member 221 cannot receive an online share transfer. Therefore, the default transfer method of paper mail is required of member 225.

Similarly, when a member with a need is setup to receive online share transfers, but a member allocated to send the share is not setup for online share transfers, then the receiving member may be inconvenienced. Member 231 has medical need 232. Member 231 is setup to receive shares via an online transfer service, as indicated by icon 233. Member 235 is not set up for online transfer service, as indicated by icon 236. Member's 235 share is allocated by the allocation engine to Member's 231 medical need 222. Member 235 sends the share 238 by writing and mailing a check. Member 231 receives the share 238 through the paper mail service. Member 231 would have received share 238 sooner if the share sending member 235 was setup to send through an online share transfer service. Even though member 231 is setup to receive online transfer service through computer network connection 239, member 235 cannot send an online share transfer. Therefore, the default transfer method of paper mail is required of member 235.

When a member with a need is not setup to receive online share transfers and a member allocated to send the share who is also not setup for online share transfers, then neither party is inconvenienced. Member 241 has medical need 242. Member 241 is not setup to receive shares via an online transfer service, as indicated by icon 243. Member 245 is also not set up for online transfer service, as indicated by icon 246. Member's 245 share is allocated by the allocation engine to Member's 241 medical need 242. Member 245 receives a paper share slip. Member 245 sends the share 248 by writing and mailing a check. Member 241 receives the share 248 through the paper mail service and not computer network 249.

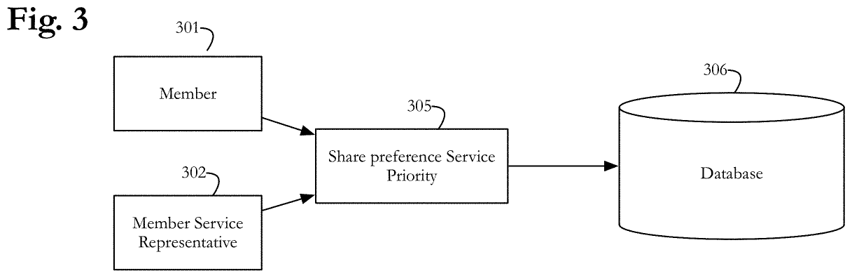

Members may select one or more preferred online transfer services to send shares and to receive shares. As shown in FIG. 3, the member 301 or the member service representative 302 may select a share preference service priority 305, which is stored in database 306. The category order for share preference service priority 305 may be a selected electronic transfer service (“e-service”), then online share slip, then paper checks. In one embodiment, the selected e-service is a single service provider. In another embodiment, the selected e-service has multiple service provider options.

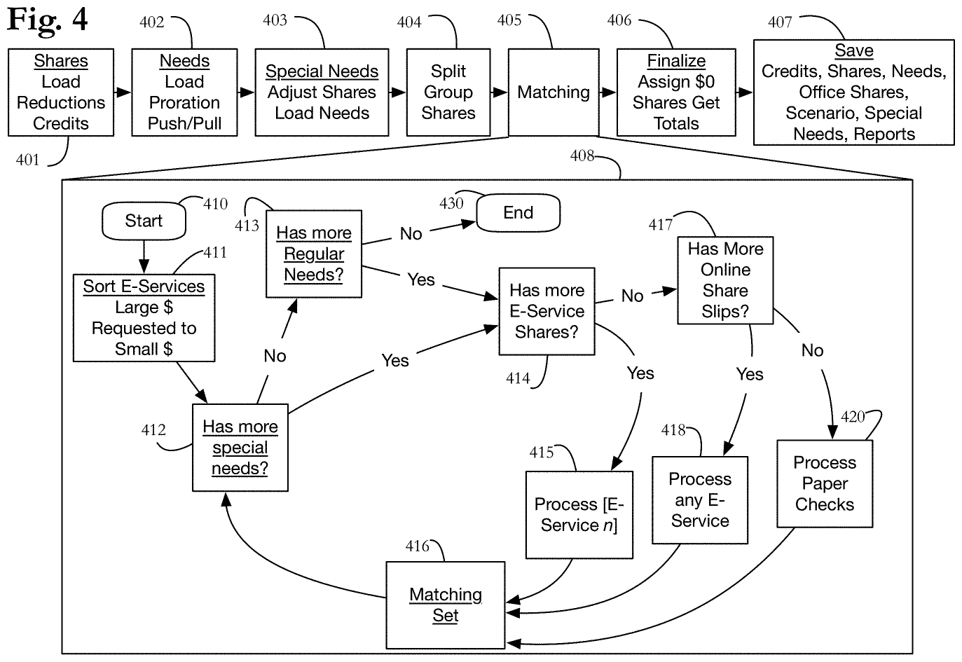

In order to increase the efficiency of sharing between members with a matching or compatible need sharing transfer service preferences, the allocation engine preferentially matches members who have expressed a preference to similar electronic transfer services together. In order to increase the percentage of members successfully match, the allocation engine prioritizes the matching of the electronic transfer service having the largest dollar amount of total needs to shares matching the electronic transfer service, as shown in FIG. 4.

In one embodiment, the first step is to load shares, as shown in step 401. All shares are loaded into a shares-analysis queue in the analysis engine. Any reductions are applied to the shares once they have been loaded. Reductions occur when the total dollar amount of available shares exceeds the total dollar amount of needs. The result is that the shares—with the exception of shares sent to the sharing organization for administrative overhead—are reduced by a predetermined percentage. Credits are also applied to the shares at this step. Credits can be applied to shares to reward certain member behavior, such as referring other members to the sharing organization. Both reductions and credits reduce the dollar amount that a member sends in their share. A credit reduces the dollar amount of a specific member's share, whereas the reduction is applied to most or all shares.

The next step is the needs step, as shown in step 402. All needs for the current period are loaded into a needs-analysis queue. At this time the total amount that is shareable is calculated. Proration is a reduction of each need by a predetermined percentage, which may happen when the total dollar amount of needs exceeds the total dollar amount of shares. Proration affects the total amount that is shareable.

The allocation engine calculates and displays the total dollar amount of shares available for the current period, the total dollar amount of loaded needs for the current period, and the total dollar amount of needs set to load in the subsequent period. The analyst can pull certain needs, or a portion thereof, from the subsequent period into the current period. Alternatively, the analyst can push certain needs, or a portion thereof, from the current period into a subsequent period. This provides the analyst with the discretionary ability to affect the total dollar amount of loaded needs for a current period. By pulling one or more additional needs into the current period, when that need would otherwise not be loaded until the subsequent period, the total dollar amount of the needs for the current period increases. This may be advantageous to fully utilize the total dollar amount of the shares for the current period. Another advantage to pulling needs into the current period is to reduce the number of shares needed for the subsequent period, thereby preventing a situation where the total dollar amount of the needs in a subsequent period exceeds the total dollar amount of the shares available in that period. A need added to the push list reduces the total amount shareable for the current period. An advantage to pushing a need to a subsequent period allows the needs in the current period to be more fully shared when the total dollar amount of needs in the current period exceed the total dollar amount of shares available in that period. The analyst can push an entire share, a specific dollar amount, or a predetermined percentage. If the total dollar amount of a need is less than a defined minimum shareable amount, then that need is removed from the need queue.

In the embodiment shown in FIG. 4, the health care sharing organization can utilize a separate sharing structure for special needs, as shown in step 403. This separate sharing structure allows members to voluntarily elect to participate in the sending and receiving of shares related to special needs. In one example, regular needs are limited to $250,000.00 and a special need is a need whose total dollar amount exceeds $250,000.00, specifically the amount of the need that exceeds $250,000.00. If the member elects to participate in the special needs program, then that member shares in the portion of a need that exceeds $250,000.00 with other members who have elected to participate in the special needs program. The member participating in the example special program only sends this additional share if there are qualifying needs. If the special program shares are not needed, the participating member holds the funds in a reserve to build up a special program participation balance. This balance can grow over the years if there the total dollar amount of the qualifying special needs is less than the cumulative set aside for the participating members over a given period. In this example, the member commits to set aside a particular amount of money per year, as determined by the program guidelines to contribute an additional share portion to the periodic share for special needs. Any special needs that have been approved for sharing are loaded into a special needs queue. In one embodiment, the allocation engine calculates the amount of special needs relative to the number of members participating in the special needs program to form a special needs participation share amount. The special needs participation share amount is added to the share of each participating member as an adjusted share amount.

Each membership or group of memberships can choose to send more than one share each period, resulting in a split share, as shown in step 404. The member service representative may associate a split share parameter with a membership willing to send multiple shares. The allocation engine generates a number of smaller shares based on the split share parameter corresponding with the number of shares the member is willing to send. The split calculator creates copies of the spilt share and adjusts the share amount accordingly, so the two shares may be of unequal amounts but will add up to the same dollar amount as a single share for that member. For large share groups there is a configurable maximum share dollar amount that will automatically force a share to be split, such that the share amount of each split share does not exceed the maximum share dollar.

The shares in the shares-analysis queue are then allocated to the needs in the needs-analysis queue in matching step 405. The matching step 405 is expanded in box 408. On the start 410, the allocation engine begins the matching step 405. The allocation engine sorts all needs marked as e-service, pursuant to step 411. Needs are sorted from largest dollar amount to smallest dollar amount. Except, however, a collector need may be added to the bottom of the queue otherwise out of the queue's order. Alternatively, the needs may be sorted by chronology, severity of a need, manually by the analyst, or according to a calculated metric that measures a member's involvement within the ministry, such as length of membership or timeliness of sending shares.

The allocation engine may prioritize the processing of special needs, as shown in step 412. The allocation engine processes needs in the special needs group with shares in the special needs group. In this example, only members who are in special needs program share to a qualifying special need. Therefore the special needs are processed in separate cycles. In another example, the allocation engine does not discriminate between a special needs member and a non-special needs member. In this first round, special needs program shares are matched to special needs in the same way that regular shares are matched to regular needs, which is discussed below.

Once all the special e-service needs are matched, the allocation proceeds to match regular e-service needs with regular shares, according to step 413. The allocation engine selects the largest regular e-service need from the needs-analysis queue. The system may look for the smallest regular e-service share that is greater than the need amount that has not yet been met. If no share from the share analysis queue will meet the remaining need amount completely, the largest share available in the share analysis queue is selected.

Once there is a preliminary match between a share and a need, the allocation engine performs matching quality controls. The system prevents a share that belongs to the need's membership from being assigned to the need. This prevents a membership from sharing with itself. For split shares, the system is configured to assign only one share per membership or group to a selected need. This prevents a membership from being asked to send two shares to the same member.

After a preliminary match and passing the error controls, the selected share is then assigned to the selected need and the amount needed is reduced by the share dollar amount. Then the share is removed from the shares-analysis queue.

If there are additional e-service shares, as shown in Step 414, the system continues to apply e-service shares from the shares-analysis queue to the selected need from the same e-service group, according to step 415. The selected share is then assigned to the selected need and the amount needed is reduced by the share dollar amount, as shown in step 416. Then the share is removed from the shares-analysis queue.

The system continue to match e-service shares, steps 413-416, to the selected need until the total amount of shares is greater than or equal to the need amount. If the total amount of the shares is greater than the need amount, the difference between the need amount and the sum of the shares is considered overage. The system searches the shares-analysis queue to ensure no shares are available that are smaller than the overage. If there is such a share available, the allocation engine removes the last added share putting the last added share back into the shares-analysis queue to be assigned to a different need.

Once the shares-analysis queue is exhausted of a selected e-service group shares, then the system proceeds to the next e-service group having the next largest dollar amount of needs. The allocation engine iteratively processes each e-service group from the largest dollar amount to the smallest dollar amount, until either the selected e-service group's needs or shares is exhausted.

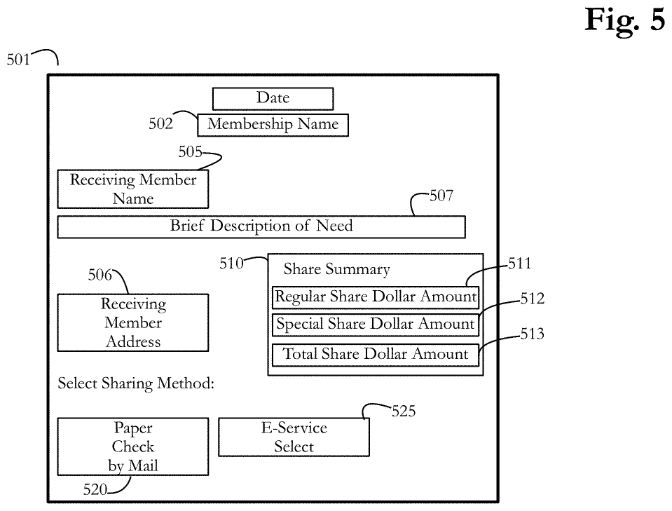

Once the e-service group needs have been matched with the e-service group shares, then the allocation engine matches any non-regular share in the shares-analysis queue to memberships that receive an online share slip, according to step 417. The online share slip 501 is an electronic graphical user display associated with a specific membership, as shown in FIG. 5 and discussed below. The allocation engine proceeds to match any remaining e-service needs with shares from the online share slip group, according to step 418. Members who receive online share slips are presented with clickable web links for e-services, and therefore may participate in the receiving member's preferred e-service even if the sending member has not indicated their participation in that e-service.

Once the queue has been exhausted of either all of the e-service needs from the needs-analysis queue or the online share slip shares from the shares-analysis queue, then the system proceeds to process paper checks, according to step 420.

The allocation engine continues to process needs from the needs-analysis queue and shares from the shares-analysis queue until all shares are assigned or all needs are met, according to step 430. The analyst may adjust various settings, such as pulls and pushes, until all the shares have been assigned and only the collector need is not met. Once the analyst is satisfied with the allocation, the analyst selects to commit the allocation to production. The allocation data is pushed to the production system to generate share slips, both printed share slips and online share slips. The system proceeds to finalize any remaining shares as shown in step 406. The system finalizes the allocation by recording and saving credits, shares, needs, shares designated as office shares, as shown in step 407. Different scenario run details are recorded. Special needs, those need s that may not fit within the normal sharing guidelines can be addressed. And reports can be run showing the statistics of the allocation scenario.

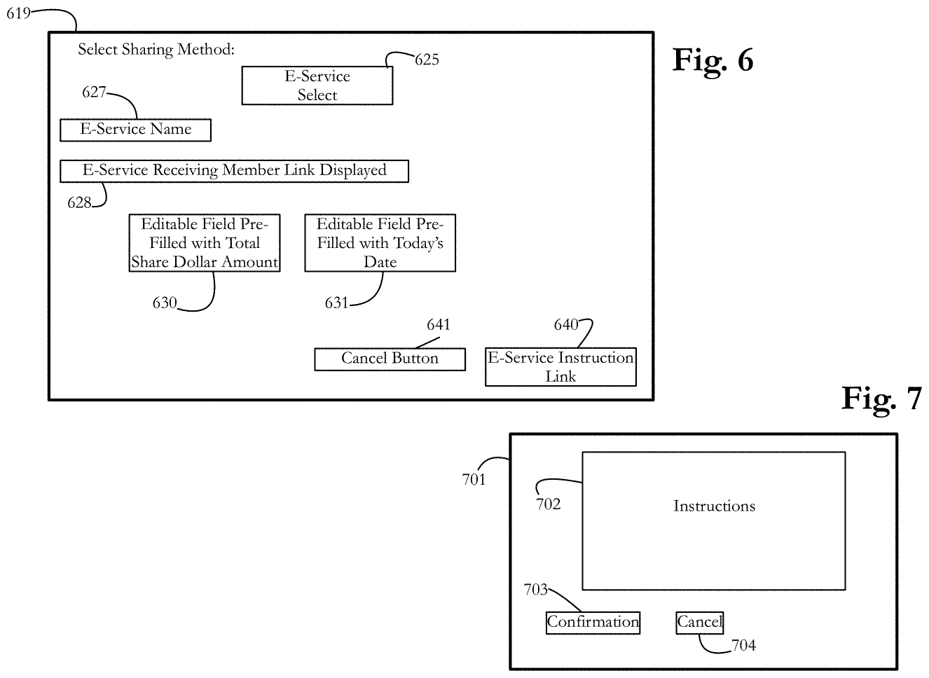

The online share slip 501 gives share information for the member's assigned share, as shown in FIG. 5. The online share slip 501 may indicate the receiving member's name 505, receiving member's mailing address 506, and a brief description of the need 507. The online share slip 501 displays a share summary 510. The share summary 510 includes the regular share dollar amount 511, the special share dollar amount 512, and a total share dollar amount 513. The online share slip also includes a clickable web link for the sending member to indicate their intention to send the share through paper mail or an e-service. The sending member selects either the paper check by mail select 520 or the e-service select 525. When the member clicks on paper check by mail select 520, a text field allows the sending member to transpose a check number and confirm that the share has been sent via postal mail. If the member clicks on the e-service select 525, a confirmation portion is displayed with pre-filled information. As shown in FIG. 6, the confirmation portion 619 shows the E-Service select 625 as having been selected. The selected e-service name 627 is displayed to confirm to the user that the appropriate e-service is selected. The URL for the receiving member page 628 with the selected e-service is displayed. The member is responsible to confirm payment sent, in a similar fashion to confirming sending the postal mail. The total share dollar amount is prefilled in an editable total share dollar amount field 630. The current date is prefilled in an editable current date field 631. The sending member is also presented with a subsequent web link 640. Activation of subsequent web link 640 causes instructions to be displayed for the selected e-service. If the member has selected the wrong e-service or otherwise wishes to cancel sending the share, there is a cancel button 641. Clicking the subsequent web link 640 may open up an instruction display 701, as shown in FIG. 7. The instruction display 701 presents the share sending member with instructions 702 specific to the selected e-service platform and how to return to the sharing organization member portal to confirm electronic transfer of the share. If the member understands and agrees with the instructions, there is a confirmation button 703. If the member does not agree with the instructions, the member may still cancel the transfer 704. In this embodiment, the confirmation button 703 is the clickable web link that sends the share sending member to the e-service portal to send the share to the receiving member. The URL destination associated with the confirmation button 703 may be hard coded to pre-fill in the total share dollar amount. Members then send their share payment through the linked e-service. The sharing member may also send a brief note of encouragement to the Member in need assigned to them.

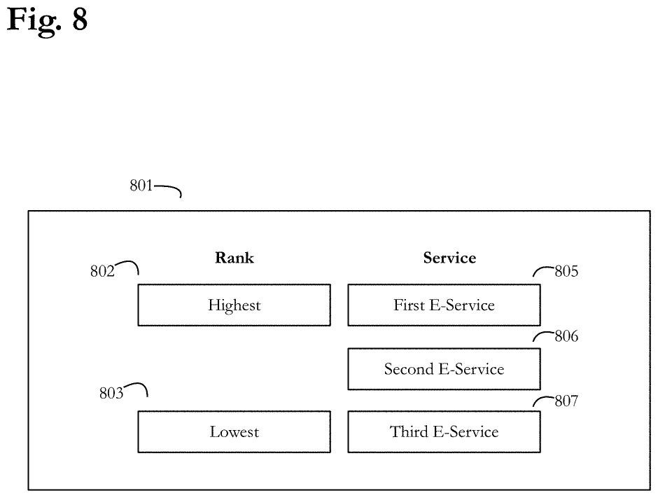

As shown in FIG. 8, the system may employ a user interface preference selection page 801 to allow the member or a member service representative to rank the service provider parameter. The member or member service representative may select a priority rank, from highest 802 to lowest 803 for individual electronic share transfer services such as first service provider 805, second service provider 806, and third service provider 807.

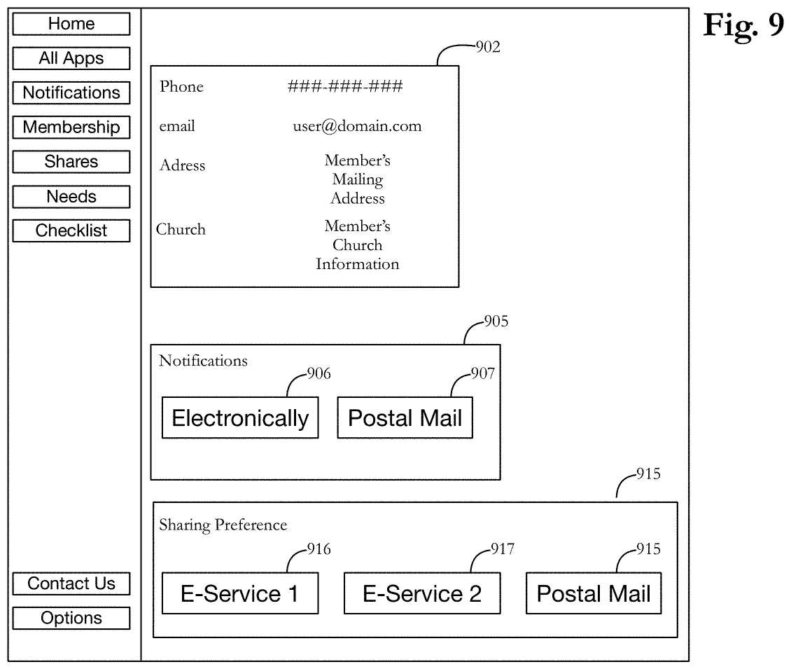

Alternatively, as shown in FIG. 9, the member accesses a graphical user interface 901 to select their e-service preference. Graphical user interface 901 displays member biographical data 902, such as member's name, address, email, phone number, and church affiliation. A notification section 905 allows the member to select between electronic communication preference 906 and postal mail communication preference 907. A sharing preference pane 915 allows the member to select a preferred e-service: a first e-service 916, a second e-service 917, and postal mail 918. When a member selects an e-service, the member is prompted with a subsequent screen to enter the e-service account profile information. For example, the member may enter their paypal.me link. The link information is used to generated a clickable web link when the member has a need shared to a sending member also enrolled in the e-service or who opts to receive electronic share slips.

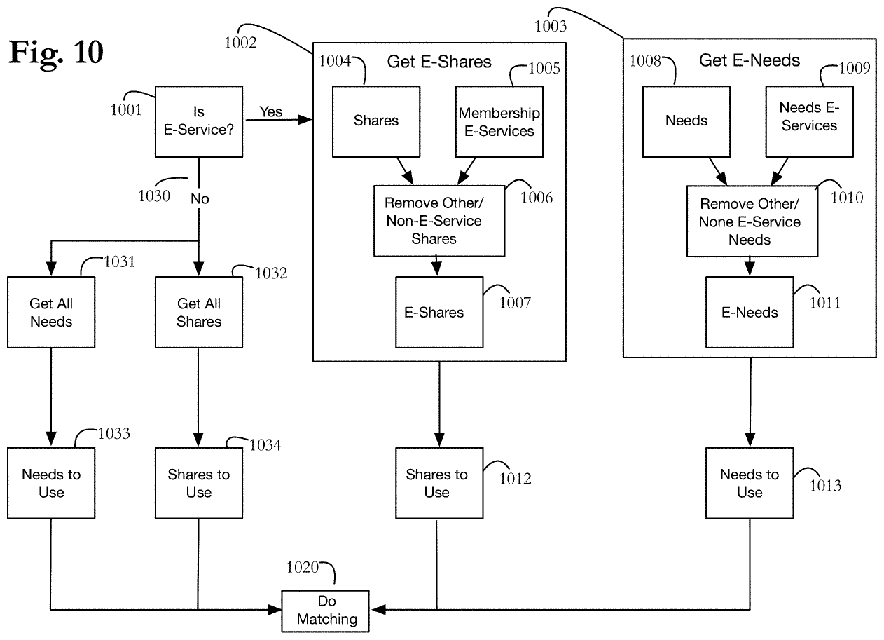

The queuing process for e-service and regular needs and shares is shown in more detail in FIG. 10. The allocation engine determines whether each need is an e-service need and whether each share is an e-service share, according to step 1001. If yes, the allocation engine proceeds to get e-shares, according to step 1002, and to get e-needs, according to step 1003. In Step 1002, shares 1004 and membership e-services 1005 are combined. From this combination, any other non-e-service shares are removed from consideration, according to step 1006. This results in only e-shares 1007. In Step 1003, needs 1008 and needs e-services 1009 are combined. From this combination, any other non-e-service needs are removed from consideration, according to step 1010. This results in only e-needs 1011. The allocation engine selects the e-shares 1007 for processing according to step 1012. The allocation engine selects the e-needs 1011 for processing according to step 1013. The allocation engine performs the matching on these selected e-shares and selected e-needs, according to step 1020. Once the allocation begins to process regular needs or shares, the allocation engine proceeds down pathway 1030. The allocation engine gets all needs 1031, which includes regular needs and any remaining e-service needs. The allocation engine also gets all shares 1032, which includes regular shares, any remaining e-service shares, and online share slip shares. The allocation engine selects all needs 1031 for processing, according to step 1033. The allocation engine selects all shares 1032 for processing, according to step 1034. The allocation engine performs the matching on these selected all shares and selected all needs, according to step 1020.

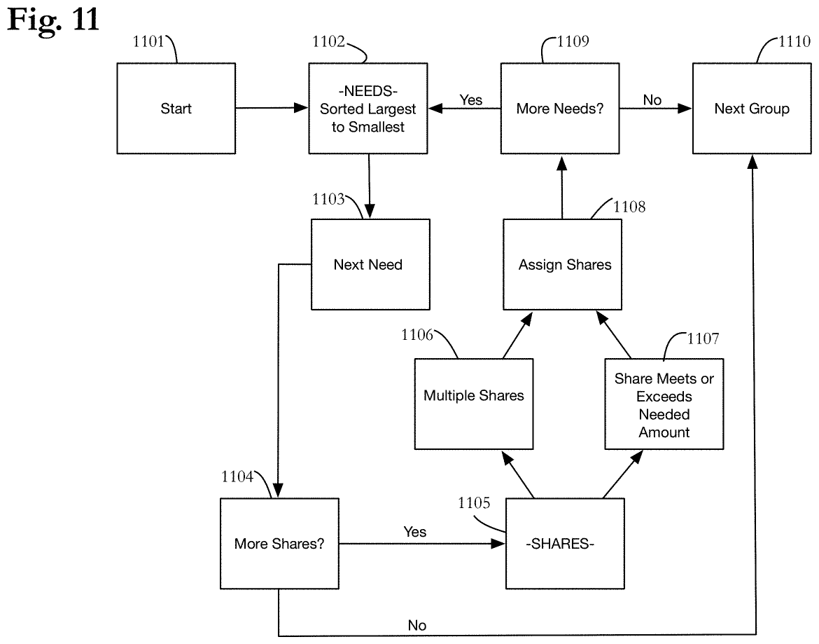

A process for matching needs is shown in FIG. 11. The same matching process may be used for both special needs and regular needs. The allocation may match the needs as separate groups, special needs with special shares, and regular needs with regular shares, by attributing an appropriate parameter for the appropriate needs and shares. Upon starting the matching process 1101, the needs are sorted by dollar amount from largest to smallest, step 1102. The largest remaining need is selected 1103. If there are more shares 1104, then the shares queue 1105 is accessed. The allocation engine attempts to match a single share that meets or exceeds the need amount 1107. If the allocation is unable to match a single share that meets or exceeds the need amount 1107, then the allocation engine makes a tentative match of multiple shares 1106. The need is either met through multiple shares 1106 or a single share that meets or exceeds the need amount 1107, and then those shares are assigned to that selected need, step 1108. The system continues to assess needs 1109. If there are more needs, then the 1102-1108 cycle continues. If there are no more needs, then the system proceeds to the next group, according to step 1110. The system may also move on to the next group if step 1104 gives a result that there are no more shares for the specified group.

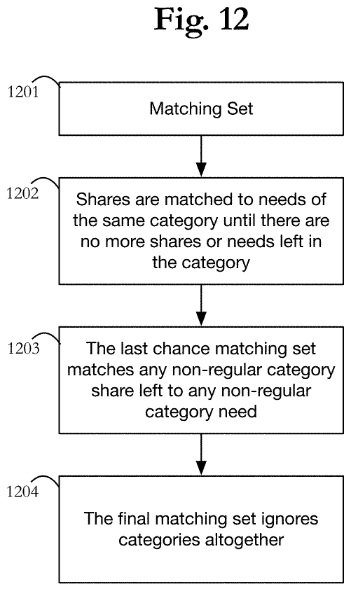

A process for a matching set 1201 is shown in more detail in FIG. 12. Shares are first matched to needs of the same category until either the shares in that category or the needs in that category are exhausted, according to step 1202. The category may be an e-service or may be another aspect of the share transaction. For example, the category may be currency, where members prefer to share in Mexican Pesos or Canadian Dollars. Needs that share the currency category may be preferentially matched with members who are setup to share in the same currency category. Additionally, electronic currency systems such as BitCoin™ and Ethereum™ combine payment networks and the form of currency. As such, electronic currency systems may be included as a category for preferential matching of needs and shares.

Once either the shares or needs of each category are exhausted, then the allocation engine may proceed to a last chance matching set, according to step 1203. During the last chance matching set, any non-regular category share is matched to any non-regular category need. In this way, more technically savvy users have a greater chance of being matched together. This last chance matching set may be more advantageous when the categories are e-services, and may be less advantageous when the categories are currencies. The final matching set ignores categories and matches any remaining need with any remaining share, according to step 1204.

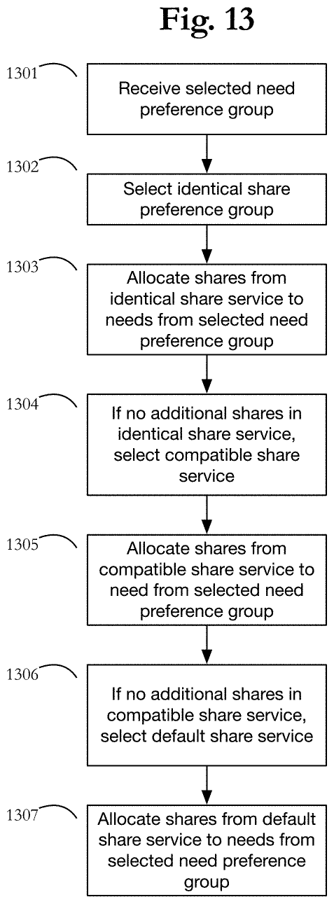

A selected need preference group may be matched with multiple compatible share preference groups, as shown in FIG. 13. The allocation engine may be configured to prioritize identical electronic share transfer services first and then proceed to secondary affinity basis preference or compatible share transfer services. The allocation engine may be configured to generate a compatible shares queue containing all of the matched sending shares or compatible sending shares from the available shares. The allocation engine first receives a selected need preference group, as shown in step 1301. The allocation engine selects needs that share an identical share service, according to step 1302. The allocation engine then proceeds to allocate shares from identical share service to the needs from the selected need preference group, according to step 1303. If no additional shares are available in the identical share service, then the allocation engine may proceed to select a matching or compatible share service, according to step 1304. Shares are allocated from compatible share service group to the needs from the selected need preference group, according to step 1305. If no additional shares are available in the compatible share service group, then the default share service group is selected, according to step 1306. Shares from the default share service group are allocated to the need from the default share service until the needs or shares are exhausted, according to step 1307.

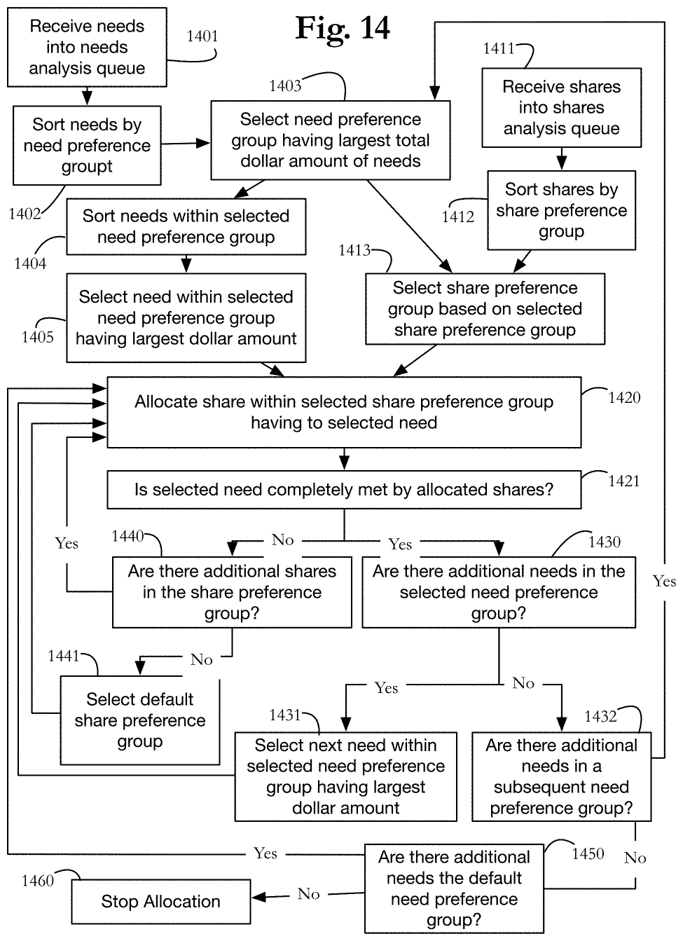

Another embodiment of the allocation engine is shown in FIG. 14. The needs qualifying for sharing under the guidelines of the sharing organization are received for a particular period into a needs-analysis queue, according to step 1401. The needs-analysis queue is sorted by need preference groups, according to step 1402. The allocation engine selects the needs preference group having the highest total dollar amount of needs, according to step 1403. The needs are sorted within the selected need preference group, from highest dollar amount to lowest dollar amount, according to step 1404. A need is selected within the selected need preference group that has the largest dollar amount, according to step 1405.

In order to assign the needs from the selected share preference group with appropriate shares, the shares for the particular period are also received into a shares-analysis queue, according to step 1411. The shares-analysis queue is sorted by share preference group, according to step 1412. The allocation engine selects the share preference group based on the selected need preference group, according to step 1413. Step 1413 can incorporate the compatible categories to preferentially select the identical (or matching) share preference group and then proceed to select a compatible preference group.

The selected need of step 1405 is then matched with shares from the selected share preference group of step 1413. The largest share within the selected share preference group is allocated to the selected need, unless the allocation engine identifies a share that is equal to or greater than the remaining share amount, according to step 1420. Alternatively, if multiple shares from the selected share preference group match or are compatible with the need, then the system may proceed to analyze a secondary affinity basis in the same way as it analyzed the primary affinity basis. If the selected need has been completely met by the allocated shares, then the allocation engine will proceed to step 1430. If the selected need has not been completely met by the allocated shares, then the allocation engine will proceed to step 1440.

If the selected need has not been completely met by the allocated shares, then the system will determine if there are additional shares in the share preference group, according to step 1440. If there are additional shares within the selected share preference group, then the allocation engine returns to step 1420 to allocate the next largest share from the selected share preference group, unless there is a single smaller share that satisfies the remaining dollar amount of the need. If there are not any additional shares within the selected share preference group, then the system selects the default share preference group according to step 1441.

If the selected need has been completely met by the allocated shares under step 1421, then the allocation engine will check whether there are additional needs in the selected need preference group, according to step 1430. If there are additional needs, then the allocation engine will select the next need within the selected need preference group having the next largest dollar amount, according to step 1431. The system then returns to step 1420 to continue the matching of the new need with the available shares. If there are no additional needs in the selected need preference group, then the allocation checks whether there are additional needs in a subsequent need preference group having the next largest total dollar amount, according to step 1432. If there are additional needs in subsequent need preference groups, then the system returns to step 1403. If there are not additional needs in a subsequent group, then the allocation engine proceeds to check whether there are additional needs in the default need preference group, according to step 1450. If there are needs in the default need preference group, then the system returns to step 1420 to assign shares without regard to preference groupings. If there are no additional needs in the default need preference group, then the system stops the allocation process according to step 1460.

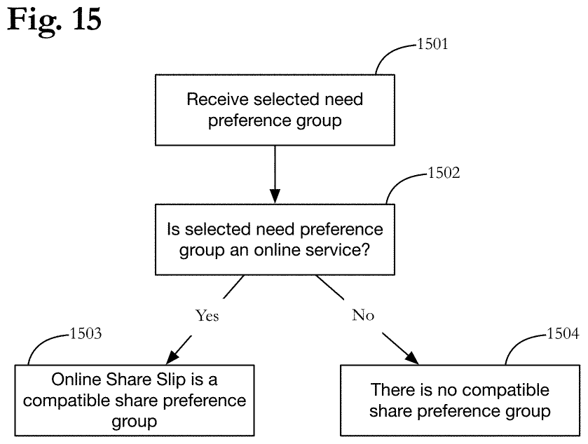

A process for determining compatible share services is shown in FIG. 15. First, the allocation engine receives a selected need preference, according to step 1501. The system determines whether the selected need preference group is an online service, according to step 1502. If the share preference group is an online service, then online share slip group is a compatible share preference group, according to step 1503. If the share preference group is not an online service, then the online share slip group is not a compatible share preference group, according to step 1504.

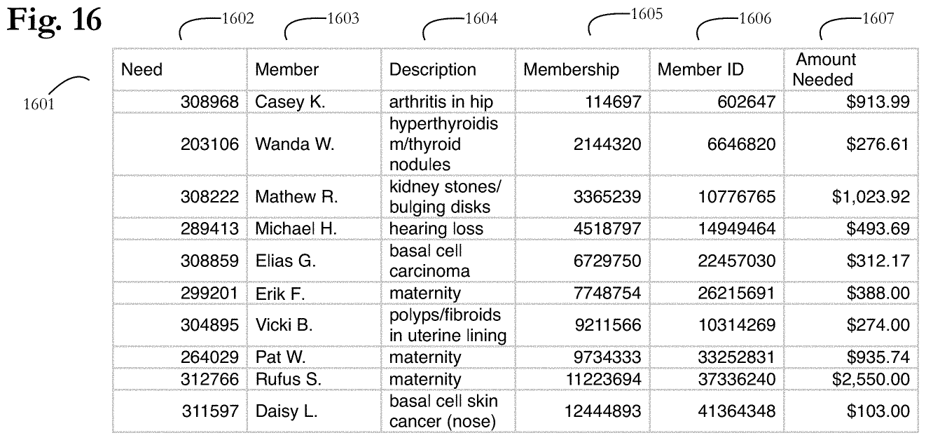

FIG. 16 shows an example data set 1601 for need submission. Needs are given a unique need identification number 1602. Each need is associated with the submitting member 1603. The data set 1601 also includes a description of the medical need 1604, membership number 1605, member ID 1606 identifying the particular member within the membership having the need, and total need amount 1607.

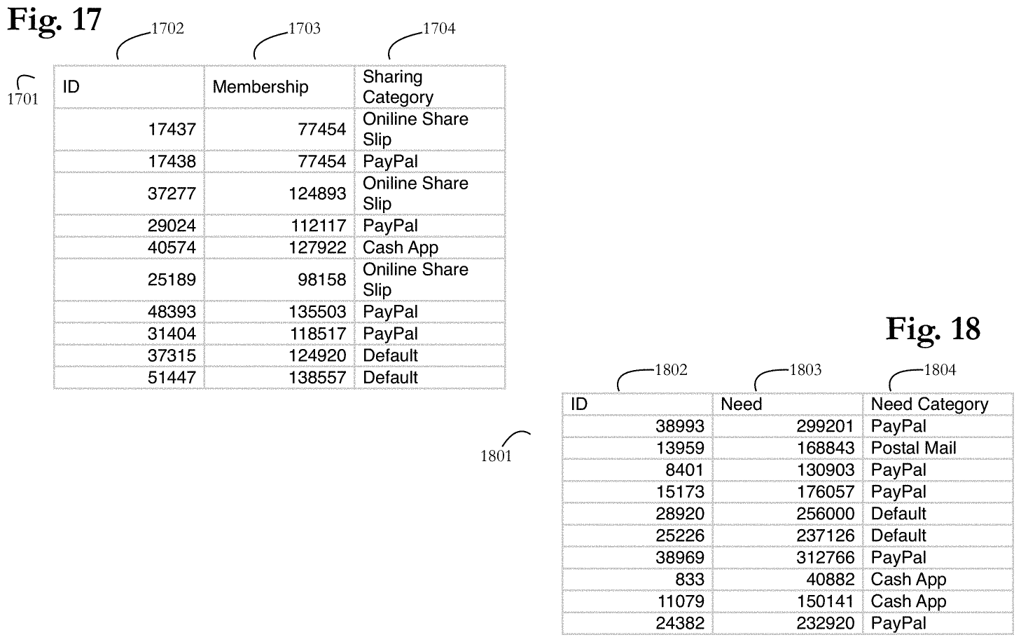

FIG. 17 shows an example sharing preference data set 1701 for storing members' sharing preferences. The data set 1701 includes a unique identification number 1702 and the membership number 1703. Each Membership is associated with one or more categories.

FIG. 18 shows an example need preference data set 1801 for storing members' need preferences. The data set 1801 includes a unique identification number 1802 and the membership number 1803. Each Membership is associated with one or more need categories.

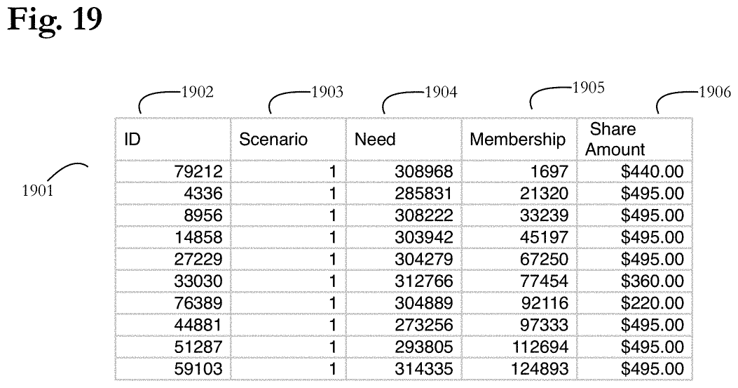

An example allocation data set 1901 from a completed run of the allocation engine is shown in FIG. 19. A unique identifier is shown in column 1902. A scenario number 1903 is recorded for each time the allocation engine is run during a period. This allows the analyst to adjust the settings and track the results the varied settings. The unique need number 1904 is included in the allocation data set 1901, along with the membership number 1905. The share amount 1906 is shown matched to the unique need number 1904. This information is used to populate the respective share slips for each member.

In order to increase the number of sharing members being connected to receiving members that have the same category, the allocation engine preferentially matches categories having the highest dollar amount. A web link is generated to direct the sharing member to an online account portal for the membership who submitted the selected need. The web link includes the share amount to reduce the opportunity for member error in transposing the share number. The online share slip is displayed to the sharing member through the online account portal.

Affinity-based allocation can be used to preferentially match sharing members with receiving members based on one or more criteria. The criteria can include location, type of medical need, household demographics, or a religious or other group affiliation.

In one example, a health care sharing organization utilizes affinity-based allocation based on location of the sharing member and the receiving member. The health care sharing organization preferentially matches a sharing member with a receiving member based on physical proximity. A physical location is associated with each member. For example, a location entry is stored as a location variable for each member. The location entry for the location variable can be the name of a country, state, county, city. The location entry for the location variable can also be a postal code (such as a ZIP code) or latitude and longitude coordinates. The location entry for the location variable can be generated dynamically, such as internet protocol (IP) address, Internet service provider (ISP), GPS coordinates, or location information generated by an app on a mobile device or a computer. The physical location can also be static, based on the physical address entered in a member's membership application. The allocation engine calculates a physical distance between a sending member and a receiving member, for example by comparing the difference between zip codes or by calculating distance from latitude and longitude of the members. The allocation engine matches shares from the shares queue that have the shortest calculated distance from the selected need. By assigning shares to members with physical proximity, the sharing member may be more personally engaged in the sharing process. A more engaged member may more quickly send a note of encouragement that comes with the sending of a share to the receiving member because of the close proximity. A more engaged member may also be quicker to send a share, driven by a sense of community.

In another example, the health care sharing organization preferentially matches a sharing member with a receiving member based on the underlying medical reason for the health care services (for example, cancer, heart disease, liver disease, kidney disease, asthma, arthritis, dementia, diabetes, epilepsy, pregnancy, broken bone, etc.) or based on the health care service itself (for example, chemotherapy, radiation therapy, blood transfusion, etc.). One or more medical description entries are associated with a medical-description bill variable. The medical diagnosis entries can correspond to a general description, a diagnosis, a billing code number, or a severity level of the medical procedure, such as severe, traumatic, or terminal illness. The sharing member may select one or more medical diagnosis entries that are personally significant, such as a disease that the sharing member personally experienced or lost a love one to. Similar to FIG. 8, the sharing member can create a medical preference list by ranking the medical reasons that are significant to the sharing member through the online account portal. The sharing member is presented with a plurality of medical diagnosis, descriptions, or categories. The sharing member ranks the plurality of medical diagnosis, descriptions or categories by ordering or assigning interest values to each of the plurality of medical diagnosis entries. In another embodiment, sharing members are attributed with an engagement rating based on the time it takes the member to send their share. Members with a fast engagement rating can be matched with needs that are more serious with a higher dollar value. This allows receiving members who are in a difficult and highly emotional time of life to receive financial support most efficiently. The health care sharing organization can flag a need as being priority, and the allocation engine is configured to match shares from the share queue from members with a fast engagement rating with needs that are marked with a priority flag. Alternatively, a plurality of medical diagnosis entries can be assigned to a sharing member automatically based on the sharing member's medical history or the medical history of the household relative of the sharing member, a process that can be performed at sign up and continually through the member's membership. By assigning medical diagnosis entries that are personally relevant to the sharing member, the sharing member may be more personally engaged in the sharing process. A more engaged member may be able to better comfort the receiving member through a note of encouragement that comes with the sending of a share. A more engaged member may also be quicker to send a share, driven by sympathy from a shared experience.

In another example of affinity-based allocation, the health care sharing organization preferentially matches a sharing member with a receiving member based on household demographics of the sharing member and the needing member. For example, a sharing member may be a part of a household with young children. The health care sharing organization could preferentially match a sharing member from a household with young children to a receiving member who is also from a household with young children. The sharing member would have knowledge of engaging a family with young children and could send a coloring book page or a favorite book along with the share for the medical expense. Alternatively, the health care sharing organization could preferentially match a sharing member from a retired household to a receiving member from a retired household. The sharing member could then write a note that was sympathetic to a medical expense related to aging. In another example, the health care sharing organization could preferentially match a sharing member that is a college age student with a receiving member that is a college age student. All of these examples serve to relationally connect the sharing member with the receiving member. Demographic preferences may include age, marital status, number of children, and occupation. Relationally connected members may be more invested in the sharing process, sharing quicker, and being more committed to mission of the health care sharing organization.

In another example of affinity-based allocation, the health care sharing organization preferentially matches a sharing member with a receiving member based on religious or other group affiliation of the sharing member and the needing member. In this example, the members would select a religious or other group affiliation through the member portal or by discussing with a customer service representative. A religious or other group affiliation is associated with each member by storing a group entry as a group variable. The group entry for the group variable can be a religion, a denomination, a church, a social club, an alumni network, a professional association, an employer, a voluntary organization. The sharing member may select one or more group entries that are personally significant. Similar to FIG. 8, the sharing member can rank the one or more group entries in an online account portal. The sharing member is presented with a plurality of group entries which may be generated by the health care sharing organization or the user enters manually. The sharing member ranks the plurality of group entries by ordering or otherwise assigning interest values to each of the plurality of group entries. Alternatively, a plurality of group entries can be assigned to a sharing member automatically based third party contributions. For example, an employer who signs up multiple members may request that the employees share with each other first. The allocation engine can be configured to match shares from the shares queue with needs from the needs queue upon matching the group entry corresponding to the share with the group entry of the selected need. The allocation engine may also be configured to recognize parent groups that contain multiple child groups. For example, a national organization may contain multiple local chapters. By selecting a local chapter, the allocation engine would first match shares from the local chapter with needs from the local chapter. The allocation engine would proceed to match shares from any local chapter under the parent group with a need selected from a local chapter of the parent group. By assigning group entries that are personally relevant to the sharing member, the sharing member may be more personally engaged in the sharing process. A more engaged member may be able to better comfort the receiving member through a note of encouragement that comes with the sending of a share. A more engaged member may also be quicker to send a share, driven by sympathy from a shared experience.

In one embodiment of the affinity-based allocation, the first step is to load shares. All shares are loaded into a shares-analysis queue in the analysis engine. All needs are loaded into a needs-analysis queue. The allocation engine then matches needs in the needs-analysis queue with shares from the shares-analysis queue. The allocation engine sorts all needs marked as affinity-based. The allocation engine is configured to prioritize certain affinity-basis. The prioritization can be based on total needs affiliated with a particular affinity-basis. The prioritization can be based on a desired efficiency: first electronic share submission and then non-electronic shares are matched based on proximity to minimize postage transportation time. The prioritization can be made to increase member engagement, by prioritizing matching based on medical need description. The prioritization could also be determined by sponsorship, allowing a third party to influence prioritization of matching based on the third party's preference. In one embodiment, the allocation engine separately addresses mandatory affinity-bases from secondary affinity-bases. Mandatory affinity-bases can be prioritized over secondary affinity-bases. For example, the health care sharing organization may agree to always share an employee member's medical bills with other employees. In such a case, the allocation engine would process the employer group entries corresponding to the mandatory affinity-bases first.

The allocation engine could then proceed to process additional secondary affinity-bases iteratively based on a priority ranking or based on total needs dollars associated with the secondary affinity-bases. By assigning priority ranking values to each of the affinity-bases, the allocation engine can maximize the number of shares from the shares queue with needs from the needs queue based on priority affinities. For example, medical history diagnoses may be a high priority affinity-basis. However, there may be a large number of shares from sending members having a cancer history but a low number of receiving members having current cancer medical bills available for matching. In that case, the allocation engine would match shares designated with a cancer medical diagnosis entry until all of the needs in the needs queue were satisfied. The allocation engine would proceed to a lower ranked affinity basis, such as household demographics. In this example, all of the needs would be matched with shares having corresponding household demographic entries.

Alternatively, the allocation engine could process secondary affinity-bases iteratively based on total needs dollars associated with each of the secondary affinity-bases. In this example, the allocation engine creates a total for each of the secondary affinity-bases. The allocation engine selects a first selected secondary affinity-basis based on needs in the needs queue having the greatest total needs amount. The allocation engine proceeds to match shares from the shares queue that correspond to the selected secondary affinity-basis. Once the allocation engine has satisfied all of the needs from the needs queue corresponding to the first selected secondary affinity-basis, the allocation engine proceeds to needs in the needs queue corresponding to the second selected secondary affinity-basis. The second selected secondary affinity-basis has the second greatest total needs amount. The allocation engine proceeds to match shares from the shares queue with needs from the needs queue, maximizing the number of sending members that are affinity matched to a receiving member.

In one embodiment, the allocation engine first matches shares from sending members who have elected to send shares electronically with needs from receiving members who have elected to receive shares electronically. For these electronically transmitted shares, the allocation engine would perform affinity-based allocation based on type of medical need. The allocation engine proceeds to match shares from the share queue with needs from receiving members who have not elected to receive shares electronically—or in situations where there are no remaining shares from members who have elected to send shares electronically. For these non-electronically transmitted shares, the allocation engine performs an affinity-based allocation based on geographic proximity.

The online share slip can display the criteria used to match the share with the need. For example, the online share slip can display the group affiliation that drove the connection: “This month, you're sharing with a fellow XYZ group member” or “Your share is staying right here in Anytown.” By displaying the criteria, the sending member is reinforced in the both the medical needs sharing community and the community driving the affinity-based allocation.

In another embodiment, third parties can request that sending members associated with their organization be preferentially matched. For example, a member organization or a third party (such as a news magazine or a host conference) may reinforce its community perception and affiliation with the needs sharing organization by sponsoring an affinity-based allocation. The online share slip can display “This month, you're sharing with a fellow XYZ conference attendee.”

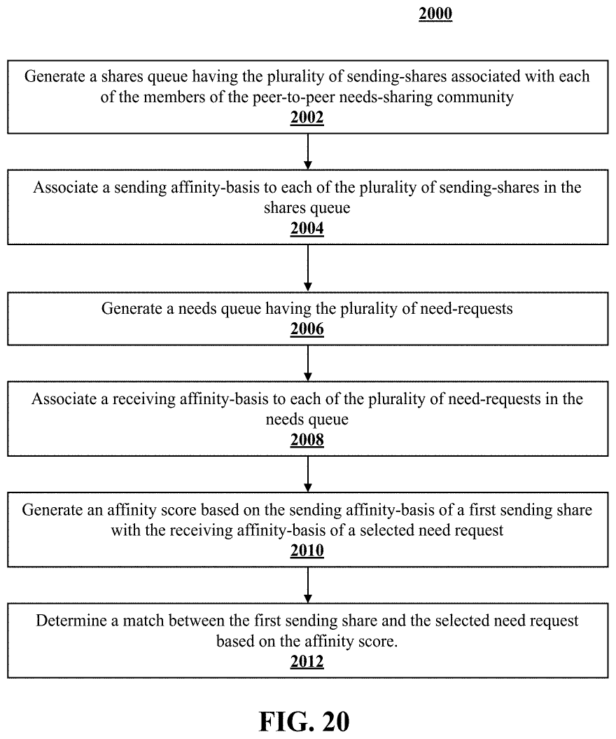

As shown in FIG. 20, a process is disclosed for generating a share slip for each member of a peer-to-peer needs sharing community 2000. Process 2000 uses an affinity-based allocation. As shown in step 2002, a shares queue is generated having a plurality of sending shares associated with each member of the peer-to-peer needs sharing community. As shown in step 2004, a sending affinity-basis is associated with each of the plurality of sending shares in the shares queue. As shown in step 2006, a needs queue is generated having a plurality of need requests from a plurality of needing members of the peer-to-peer needs sharing community. As shown in step 2008, a receiving affinity-basis is associated with each of the plurality of need requests in the needs queue. As shown in step 2010, the plurality of sending shares is allocated to the plurality of need requests in an iterative process. In step 2010, an affinity score is generated based on the sending affinity-basis of a first sending share with the receiving affinity-basis of a selected need request. In step 2010, a match is determined between the first sending share and the selected need request based on the affinity score. As shown in step 2012, the share slip is then generated for each member of the peer-to-peer needs sharing community.

Step 2010 can optionally include the calculation of a distance value between the first sending share and the selected need request. Step 2010 can optionally include selecting the match based on the affinity score corresponding to a shortest distance value. Step 2010 can optionally include accessing a table listing the sending affinity-basis that are matched or compatible with the receiving affinity-basis. Step 2010 can optionally include differentiating the affinity score for comparisons where the share affinity-basis is equal to the need affinity-basis from comparisons where the share affinity-basis is different from the need affinity-basis. Step 2010 can optionally include selecting a match from a highest sending share having a highest share value if two or more of the plurality of sending shares in the shares queue have a respective affinity score that are equal.

Process 2000 can optionally include the step of printing paper share slips for paper check members and delivering paper share slips to the paper check members by postal mail.

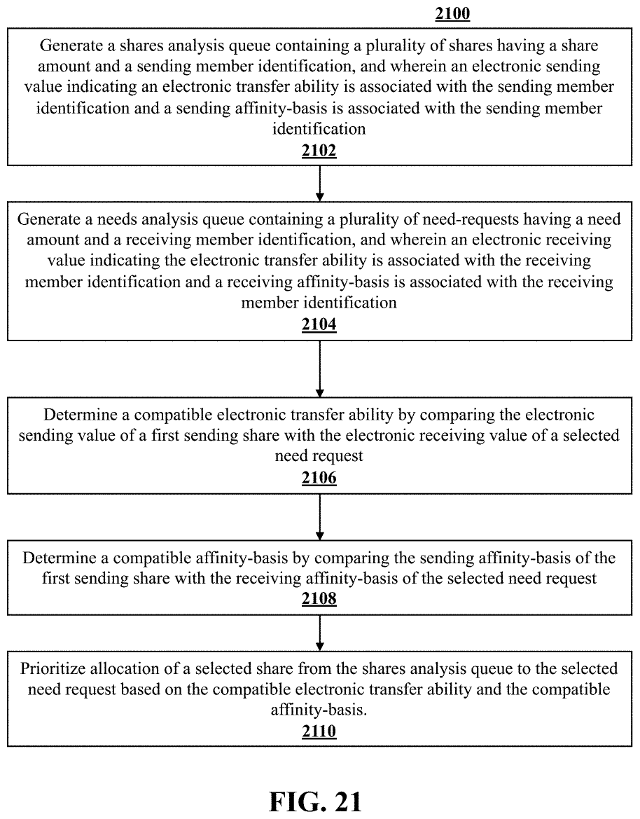

As shown in FIG. 21, a process for allocation of shares to needs within a peer-to-peer needs sharing organization is disclosed as process 2100. As shown in step 2102, a shares analysis queue is generated containing a plurality of shares having a share amount and a sending member identification. An electronic sending value indicating an electronic transfer ability is associated with the sending member identification. A sending affinity-basis is associated with the sending member identification. As shown in step 2104, a needs analysis queue is generated containing a plurality of need-requests having a need amount and a receiving member identification. An electronic receiving value indicating the electronic transfer ability is associated with the receiving member identification. A receiving affinity-basis is associated with the receiving member identification. As shown in step 2106, a matching or compatible electronic transfer ability is determined by comparing the electronic sending value of a first sending share with the electronic receiving value of a selected need request. As shown in step 2108, a matching or compatible affinity-basis is determined by comparing the sending affinity-basis of the first sending share with the receiving affinity-basis of the selected need request. As shown in step 2110, the allocation of a selected share from the shares analysis queue to the selected need request is prioritized based on the compatible electronic transfer ability and the compatible affinity-basis.

Optional to process 2100, a first clickable web link can be generated and encoded with an identification of a selected receiving member associated with the selected need request and the share amount and configured to direct a sharing member associated with the selected share to an online payment portal. Optional to process 2100, a clickable weblink is displayed through an online account portal to the sharing member. Optional to process 2100, a plurality of affinity-bases is displayed through the online account portal to a selecting member. The selecting member associates a priority-ranking associated with the selecting member based on the selecting member order the plurality of affinity-bases through the online account portal. Optional to process 2100, an affinity-basis compatibility database is accessed, the affinity-basis compatibility database associates unique affinity-bases based on group affiliations. The allocation engine determines the matching or compatible affinity-basis based on a lookup to the affinity-basis compatibility data. Optional to process 2100, the compatible affinity-basis is determined based on third party sponsorship.

It is understood that other embodiments will become readily apparent to those skilled in the art from the following detailed description, wherein various embodiments are shown and described by way of illustration only. As will be realized, the concepts are capable of other and different embodiments and their several details are capable of modification in various other respects, all without departing from the spirit and scope of what is claimed as the invention. Accordingly, the drawings and detailed description are to be regarded as illustrative in nature and not as restrictive.

1. A process for allocating a plurality of sending shares to a plurality of need requests for members of a peer-to-peer needs-sharing community, the process comprising the steps of:
a. generating a shares queue having the plurality of sending shares associated with each of the members of the peer-to-peer needs-sharing community;
b. associating a sending affinity-basis to each of the plurality of sending shares in the shares queue;
i. wherein the sending affinity-basis associated with each of the plurality of sending shares is based on a medical history of a household relative of a respective member of the peer-to-peer needs-sharing community;
c. generating a needs queue having the plurality of need requests;
d. associating a receiving affinity-basis to each of the plurality of need requests in the needs queue;
i. wherein the receiving affinity-basis is based on a medical description of the selected need request; and
e. allocating a first sending share to a selected need request if the sending affinity-basis of the first sending share matches the receiving affinity-basis of the selected need request.

2. The process of claim 1, further comprising:
a. generating a compatible shares queue comprising a selection of the plurality of sending shares whose sending affinity-basis matches the receiving affinity-basis of the selected need request;
b. calculating a distance value between each sending share in the compatible shares queue, wherein the distance value corresponds to a physical distance between a first location of a first sending member associated with the first sending share and a second location of a first receiving member associated with the selected need request; and
c. wherein the step of allocating the first sending share to the selected need request further depends upon:
i. selecting a first sending share having a shortest distance value.

3. The process of claim 1, wherein the step of allocating the first sending share to the selected need request further comprises:
a. accessing a table listing a plurality of sending affinity-bases that are compatible with the receiving affinity-basis.

4. The process of claim 1, further comprising:
a. if a plurality of matched sending shares have a respective sending affinity-basis that matches the receiving affinity-basis of the selected need request, then proceed to a second comparison comparing a secondary affinity basis between the plurality of matched sending shares and the selected need request.

5. The process of claim 1 further comprising the steps of:
a. printing paper share slips for paper check members; and
b. delivering paper share slips to the paper check members by postal mail.

6. The process of claim 1, further comprising:
a. if a plurality of matched sending shares have a respective sending affinity-basis that matches the receiving affinity-basis of the selected need request, then assigning a first sending share from the plurality of matched sending shares having a highest share value.

7. (canceled)

8. (canceled)

9. (canceled)

10. A process for allocating shares to needs within a peer-to-peer needs sharing organization, the process comprising:
a. generating a shares analysis queue comprising a plurality of shares, each of the plurality of shares having a share amount, a sending member identification, and a sending affinity-basis;
b. assigning the sending affinity-basis for each of the plurality of shares based on a medical preference list of a sending member associated with each of the plurality of shares;
c. generating a needs analysis queue comprising a plurality of need requests, each of the plurality of need requests having a need amount, a receiving member identification, and a receiving affinity-basis;
i. wherein the receiving affinity-basis relates to an underlying medical reason for a health care service associated with the selected need request; and
d. allocating a first sending share from the shares analysis queue to a selected need request from the needs analysis queue if the first sending share has a first affinity basis compatible with a selected receiving affinity-basis of the selected need request.

11. The process of claim 10, further comprising the step of:
a. generating a first clickable web link encoded with an identification of a selected receiving member associated with the selected need request and a selected share amount, the first clickable web link is configured to direct a first sharing member associated with the first sending share to an online payment portal.

12. The process of claim 11, further comprising the step of:
a. Displaying a clickable weblink through an online account portal to the first sharing member.

13. The process of claim 12, further comprising the step of:
a. displaying a plurality of affinity-bases through the online account portal to a selecting member; and
b. associating a priority-ranking of the plurality of affinity-bases through the online account portal as ranked by the selecting member.

14. The process of claim 10, further comprising the step of:
a. accessing an affinity-basis compatibility database associating a first sending affinity-basis with a first receiving affinity-basis based on a group affiliation; and
b. determining a compatible affinity-basis based on a lookup to the affinity-basis compatibility data.

15. The process of claim 10, further comprising the step of:
a. determining a compatible affinity-basis based on third party sponsorship.

16. The process of claim 10, wherein the sending affinity-basis indicates a payment sending ability and the receiving affinity-basis indicates a payment receiving ability.

17. (canceled)

18. The process of claim 10, further comprising the step of:
a. assigning the sending affinity-basis for each of the plurality of shares based on a medical history of a sending member associated with each of the plurality of shares.

19. (canceled)

20. The process of claim 10, further comprising the step of:
a. updating the sending affinity-basis associated with a selected receiving member based on the underlying medical reason for the health care service associated with the selected need request.
